(19) United States
(12) Patent Application Publication
(10) Pub. No.: US 2009/0246765 A1
(43) Pub. Date: Oct. 1, 2009

(54) METHOD AND DEVICE FOR DETECTING TARGET SUBSTANCE USING PROBE CARRIER

(75) Inventor: Takahiro Suita, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/095,502

(22) PCT Filed: Nov. 30, 2006
(86) PCT No.: PCT/JP2006/324392

§ 371 Date: May 29, 2008

(30) Foreign Application Priority Data
Dec. 1, 2005 (JP) 2005-348011

(51) Int. Cl. C12Q 1/68; G01N 33/566; C12M 1/34; G01N 21/76

(52) U.S. Cl. 435/6; 435/7.1; 435/287.2; 435/288.7

(57) ABSTRACT

A detecting device for detecting an enzyme present on a predetermined location on a substrate using a luminescence reaction, includes a mounting stage for mounting the substrate, a spotting means for locally applying a micro-droplet substance which contributes to a luminescence reaction with the enzyme onto a predetermined location on the substrate, and a detecting means for detecting a luminescence signal generated by applying the substance based on the timing of discharging a liquid droplet.

### TECHNICAL FIELD

The present invention relates to a method of detecting a target substance using a probe carrier such as a DNA microarray in which a probe capable of specific binding to the target substance is immobilized on a substrate and to a detecting device used for the method.

### BACKGROUND ART

Detection methods using probe arrays such as DNA microarrays for diagnoses of infectious diseases, detection of cancer cells, and so on have been known in the art. In any of such methods, DNA of interest is extracted from a sample such as blood or feces and then reacted with a probe array, followed by identifying the target DNA of a reacted probe.

Conventionally, for detecting the presence or absence of such a reaction, a method of binding a fluorescent label to a target nucleic acid has been used (see, for example, Japanese Patent Application Laid-Open No. H11-187900).

Further, a chemiluminescence method has been known as a method of detecting a biological sample with high sensitivity.

Such a method of detecting a biological sample using a chemiluminescence reaction is conventionally used in an immunoassay. In the immunoassay, at first, a complex containing a target substance is formed by an antigen-antibody reaction, labeled with a substance serving as a detection marker, and then subjected to a luminous reaction to detect luminescence from the complex.

Various attempts have been conducted to utilize chemiluminescence in detection. For example, Japanese Patent Application Laid-Open No. H07-190937 discloses a device for liquid fluorescence analysis based on a chemiluminescence method, which is characterized in that flow channels branching from a chemiluminescence reagent mixer are connected with respective points on a flow channel for a liquid to be measured which is connected with a flow cell.

In addition, in WO 2003/31952, for realizing the detection of chemiluminescence on a microwell plate, there is disclosed a luminescence detecting device having means for retaining a microarray plate, a plurality of light conducting guides capable of inserting their tips into the respective microwells at the same intervals as those of microwells formed in the microarray plate; and means for injecting a substrate solution to introduce a substrate solution for a luminescence reaction into the respective microwells, which is used in combination with the light conducting guide.

### DISCLOSURE OF THE INVENTION

WO 2003/31952 includes an attempt to apply a chemiluminescence method onto a DNA microarray by which higher sensitivity than a fluorescence labeling method can be expected.

However, the above-mentioned method does not include detecting chemiluminescence using a flat substrate such as a conventional DNA microarray. In other words, the method disclosed in WO 2003/31952 includes providing a plurality of minute wells on a substrate and individually inserting luminescence detecting units into the plurality of microwells, in which each luminescence detecting unit integrates capillaries for injecting a reagent into the wells and a light conducting guide for detecting luminescence. Therefore, the method disclosed in WO 2003/31952 requires complicated constructions of the array substrate and the detecting device.

Besides, the method disclosed in WO 2003/31952 contains the procedure in providing liquid reagents to wells on a substrate. Therefore, it may cause large background emission because of the thickness of wells.

Furthermore, the method disclosed in WO 2003/31952 causes diffusion of luminescent reagents in wells. For this reason, the reaction time for the luminescent reaction could be changed on each wells respectively. Moreover, it takes much time because the method disclosed in WO 2003/31952 has to insert luminescent detecting units into the plurality of microwells respectively.

In addition, a spotting to a flat substrate may not detect correct luminescent signal by the outside emission of a probe carrier.

In consideration of the above-mentioned problem, the present invention intends to provide a method, which is capable of using the conventional flat microarray and of detecting with higher sensitive and accurate microarray in a short time compared with the conventional fluorescent detection, and a detecting device used for the method.

According to the present invention, in order to solve the above-mentioned problem, there is provided a method of detecting a target substance by a luminescence reaction using a probe carrier in which a plurality of probes capable of specifically binding to a target substance are immobilized on different locations on the surface of a substrate, including spotting liquid droplets containing a reaction-contributing substance that contributes to a luminescence reaction on each of the locations where the probes are immobilized on the probe carrier.

Further, according to the present invention, there is provided a detecting device for detecting a target substance by a luminescence reaction using a probe carrier in which a plurality of probes capable of specifically binding to a target substance are immobilized on different locations on the surface of a substrate, including: a spotting means for spotting liquid droplets containing a reaction-contributing substance that contributes to a luminescence reaction on each of the locations where the probes are immobilized on the probe carrier; and a detecting means for detecting a luminescence signal on the probe location spotted.

According to the present invention, micro-droplets each containing a substance that contributes to a luminescence reaction are spotted on the respective locations where probes are immobilized on a flat substrate, so that a chemiluminescence reaction on the surface of a carrier can be suitably detected and more accurately compared to the conventional detection method by controlling the timing of discharging a liquid droplet. Besides, it is possible to detect luminescent signal on each of the locations altogether by using area-sensor. Therefore, it becomes possible to detect a target substance with simplicity and higher sensitivity in a short time using a probe carrier such as a DNA microarray.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a schematic diagram illustrating an example of an enzyme-detecting device of the present invention;

FIG. 2 is a schematic diagram illustrating another example of the enzyme-detecting device of the present invention; and

FIGS. 3A and 3B are schematic diagrams each illustrating a signal change in a chemiluminescence reaction.

### BEST MODE FOR CARRYING OUT THE INVENTION

Preferred embodiments of the present invention will now be described in detail in accordance with the accompanying drawings.

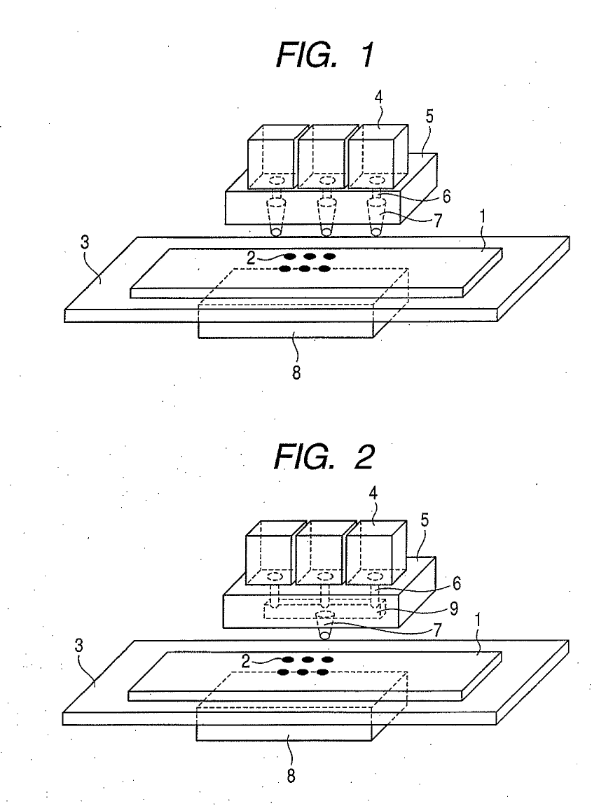

Hereinafter, one embodiment of the target substance-detecting device of the present invention will be described with reference to FIG. 1. However, the device is not limited to this embodiment.

The term “luminescence reaction” used herein may be any reaction that can be detected by luminescence and examples thereof include chemiluminescence reactions and bioluminescence reactions each using enzymes.

A substrate 1 is provided with a complex immobilized thereon. The complex contains a first biological substance (serving as a sample to be detected) such as a nucleic acid (e.g., DNA) or an antigen, and a second biological substance having a marker enzyme and being capable of specifically binding to the first biological substance. The complex can be obtained by reacting the first biological substance immobilized in a spot 2 on the substrate 1 with the second biological substance. If the second biological substance is a known substance, the first substance that specifically binds to the second biological substance can be identified. The binding of the first and second biological substances may be hybridization of complementary sequences if those biological substances are nucleic acids or may be an antigen-antibody reaction if those biological substances are an antigen and an antibody. Further, the configuration of the complex immobilized on the substrate 1 is not limited to one exemplified above as far as the configuration enables detection of the complex containing the first and second biological substances by using the marker enzyme, so that it may be different depending on the type of detection method to be used.

The substrate 1 is preferably configured so as to have a flat surface on which probes are immobilized. In general, therefore, a flat substrate is used.

The device has a mounting plate 3 on which the substrate 1 can be mounted. The substrate 1 is detachably immobilized on the mounting plate 3.

Means for spotting a substance (luminescence reaction-contributing substance) that contributes to a luminescence reaction thereof with an enzyme on the substrate 1 contains tanks 4 for storing the luminescence reaction-contributing substance and a head 5 for spotting the luminescence reaction-contributing substance on the substrate 1 by means of inkjet function. The head 5 includes discharge nozzles 7 for discharging the luminescence reaction-contributing substance and flow channels 6 for introducing the luminescence reaction-contributing substance from the tanks 4 to the corresponding discharge nozzles 7.

The means for spotting the luminescence reaction-contributing substance is preferably one with which the application amount of the luminescence reaction-contributing substance and the application time (including timing) thereof can be controlled. The head 5 is preferably an inkjet head because the inkjet head is able to apply a minute level of luminescence reaction-contributing substance within a shorter time period and the application amount of the luminescence reaction-contributing substance and the application time thereof can be easily controlled. Therefore, it is preferable to use an inkjet head as the head 5.

Further, the spotting means has means (not shown) for moving the head 5 in a two-dimensional direction with respect to the surface of the substrate 1.

Alternatively, the head 5 may be fixed while the mounting plate 3 for mounting the substrate 1 is displaced if their relative displacements can be realized.

An inkjet recording apparatus is able to carry out non-contact printing because recording is performed with ink discharged from a recording head, so that an extremely stable recording image can be obtained.

The spotting means may have any configuration as far as the means is capable of locally applying a liquid droplet onto a desired position in a desired amount and timing. For example, the spotting means may be a pin spotter or the like.

The inkjet head 5 may be any of conventionally-known inkjet heads employed in image formations with printers or the like.

An inkjet system may be either a piezo-jet system in which a piezo element (piezoelectric element) is driven to apply discharge energy to liquid in the head, or a Bubble-Jet (registered trademark) system with which thermal energy is applied.

The amount of liquid to be discharged, which can be controlled using the Bubble-Jet or piezo-jet system, is approximately in the range of about 2 to 50 picoliters. Therefore, an applying means using the Bubble-Jet or piezo-jet system is very effective for applying a minute amount of a substance on the substrate 1.

The device shown in FIG. 1 includes a plurality of (three in this case) tanks 4 and flow channels 6 and nozzles 7 are provided for the respective tanks 4, so that luminescence reaction-contributing substances can be independently discharged from the respective tanks 4. Such a configuration of the device allow the tanks 4 to independently store, for example, a luminescence reagent, a substrate, and a sensitizer and independently apply them onto the substrate 1. Such a configuration is suitable for the inkjet system.

In contrast, the device shown in FIG. 2 is configured to introduce luminescence reaction-contributing substances from a plurality of (three in this case) tanks 4 which is communicated with a plurality of flow channels 6 into a mixing vessel 9 and then discharge a resulting mixture of luminescence reaction-contributing substances from a single discharge nozzle 7. Such a configuration of the device allows the device itself to independently store, for example, a luminescence reagent, a substrate, and a sensitizer and then discharge a mixture thereof from the single discharge nozzle 7. Therefore, this configuration is suitable for the inkjet system.

In addition, the device contains a detection means 8 for detecting a luminescence signal generated by a luminescence reaction. The detection means 8 may employ, for example, a CCD or CMOS sensor.

Further, it is preferable that the detection means 8 detects a luminescence signal on a probe location provided with a liquid based on the timing of discharging the liquid droplet. It is more preferable that the detection means 8 is capable of detecting a luminescence signal on a location provided with the substrate 1 simultaneously or directly after the discharge of the liquid.

For easily detecting the luminescence level immediately after providing a luminescence reaction-contributing substance such as a chemiluminescence reagent, it is preferable to provide a substrate 1 which is a transparent substrate made of silica glass or the like and to arrange the detection means 8 so as to detect a luminescence signal from the back surface side of the substrate 1 in the case where the surface on which a luminescence reaction-contributing substance is applied is defined as a front surface side. In this case, the mounting plate 3 is configured so as to be able to receive light from the back surface side. For example, a portion of the mounting plate 3 with which at least light emitted from spots 2 is allowed to pass to the back surface side of the mounting plate 3 is made transparent, thereby allowing the back surface side to receive the light.

The detection means 8 may detect the luminescence level by using a photoelectron multiplier or connecting a sensor such as CMOS onto a substrate 1.

The luminescence reaction may be a chemiluminescence reaction or a bioluminescence reaction. The luminescence reaction-contributing substance may be at least one selected from the group consisting of a chemiluminescence reagent, a substrate used in the chemiluminescence reaction, and a sensitizer used in the chemiluminescence reaction. Alternatively, the luminescence reaction-contributing substance may be at least one selected from the group consisting of a bioluminescence reagent, a substrate used in the bioluminescence reaction, and a sensitizer used in the bioluminescence reaction.

For the chemiluminescence or bioluminescence reagent, any of conventionally-known reagents can be suitably used. Such a reagent may be, for example, luminol, isoluminol, or luciferin.

For the substrate used in a chemiluminescence or bioluminescence reaction, any conventionally-known substrate used in chemiluminescence or bioluminescence can be suitably used. Such a substrate is, for example, hydrogen peroxide.

For the sensitizer used in the chemiluminescence or bioluminescence reaction, any conventionally-known sensitizer used in chemiluminescence or bioluminescence reactions can be suitably used. For example, p-iodophenol can be used.

The luminescence reaction-contributing substance may be dissolved in an appropriate solvent for use.

The enzyme which can be used is any conventionally-known enzyme serving as a catalyst for the chemiluminescence or bioluminescence reaction. For examples, the enzymes include horseradish peroxidase, alkaline phosphatase, and luciferase.

As described above, according to the present invention, a luminescence reaction-contributing substance can be added only to a spot, which can prevent the detection of an enzyme nonspecifically absorbed on a substrate.

More specifically, the device including a tank for storing a luminescence reaction-contributing substance and a head having a nozzle for discharging a liquid droplet of the luminescence reaction-contributing substance is provided. Therefore, the liquid droplet of the luminescence reaction-contributing substance can be provided on the substrate, thereby allowing a reagent to be locally added to only a spot area of the substrate.

Further, by controlling the application amount and application time of the luminescence reaction-contributing substance, for example, an extremely minute amount of the luminescence reaction-contributing substance is locally dropped on a spot, thereby increasing the reproducibility of the luminescence level.

By using the present invention, an examination on a biological sample using a luminescence reaction can be carried out in a small amount with precision.

In the detection method of the present invention, furthermore, an enzyme nonspecifically absorbed on an area other than the spot area on the substrate is not provided with a luminescence reaction-contributing substance, so the enzyme does not affect on the detection. Therefore, there is no increase in the luminescence level of the background (nonspecific luminescence from other than probe location). The device for adding a reagent to a spot using a flow-injection method as described in Japanese Patent Application Laid-Open No. H07-190937 adds a chemiluminescence reagent, a substrate for a chemiluminescence reaction, and a sensitizer for a chemiluminescence reaction onto the substrate. In this method, the reagent is dropped onto the entire substrate. Thus, an enzyme nonspecifically absorbed on an area other than a spot area may also cause a luminescence reaction to thereby allow an increase in background chemiluminescence with a poor S/N ratio of chemiluminescence. Further, in the flow-injection method, two or more chemiluminescence reagents are simultaneously added, so the time period of luminescence reaction can be hardly controlled. Therefore, the reproducibility of luminescence level may vary in some cases.

By using the method of the present invention, it is possible to provide a method of detecting a target substance each of which employs a suitable probe carrier on a flat substrate respectively capable of preventing the detection of an enzyme nonspecifically absorbed on a substrate and increasing the S/N ratio in the enzyme detection using a luminescence reaction, and a detecting device used for the method.

### (Method of Detecting Target Substance)

Next, the method of detecting a target substance according to the present invention will be described in more detail.

The detection method of this embodiment includes the following steps of:

(1) reacting a target substance with a probe on a probe carrier;

(2) spotting a liquid droplet that contains a reaction-contributing substance, which contributes to a luminescence reaction, on a location where a probe on the probe carrier is immobilized; and

(3) detecting a luminescence signal on the location where the probe is immobilized.

Here, each of the above-mentioned steps will be described in detail.

(1) Step of Reacting Target Substance with Probe on Probe Carrier

Prior to the step of spotting a liquid droplet containing a reaction-contributing substance which contributes to a luminescence reaction onto a probe carrier, a target substance is subjected to a reaction with a probe on the probe carrier. The probe carrier which can be used in the present invention has been already described above. In this embodiment, an example in which the target substrate is DNA will be described. In this step, DNA of interest extracted from a sample such as blood or feces is reacted with a probe carrier. For the reaction, treatment conditions of extracting the DNA of interest from the sample, the amplification of the DNA, the reaction of the DNA with the probe carrier (hybridization reaction), and so on may employ the conventional method (e.g., a method as described in Japanese Patent Application Laid-Open No. H11-187900).

Note that it is preferable to combine the target nucleic acid with a label that allows a subsequent chemiluminescence detection. For example, a method in which an enzyme such as biotin may be combined with the target nucleic acid or a method in which an enzyme-labeled probe for sandwich assay may be combined with the target nucleic acid by hybridization reaction in advance is employed.

After the above-mentioned reaction step, DNA or the like that do not react with the probe on the substrate may be removed by washing. However, unlike the conventional fluorescence detection, luminescence from around the spot is not observed, so any operation like the one conventionally carried out, such as a blocking treatment, will not be required.

(2) Step of Spotting Liquid Droplet that Contains Reaction-Contributing Substance, Which Contributes to Luminescence Reaction, on Location Where Probe on the Probe Carrier is Immobilized

Next, a luminescence reagent is spotted on a location (spot location) where a probe is immobilized on an array. The timing of spotting the luminescence reagent on each spot and the amount of the reagent to be spotted on each spot may be individually controlled. It is preferable, however, that the spotting is simultaneously carried out on the respective spots or is sequentially carried out in an equal amount at specified time intervals.

At the probe-immobilized location where the target substance has reacted in the above-mentioned step (1), the reaction is initiated by a reaction-contributing substance and a luminescence signal is then observed. The spotting of the present invention is carried out such that a reagent in liquid droplet is discharged to the substrate, thereby forming a reagent-containing liquid droplet on the surface of the substrate. The formation of a liquid droplet on the surface of the substrate can be controlled to retain the liquid as a droplet on the substrate in consideration of the discharge rate of a liquid droplet, the composition (viscosity and surface tension) of the liquid, and the surface condition of the substrate.

Further, the spotting step may include at least one of: a first step of spotting a liquid droplet containing a luminescence reagent onto a location where a probe is immobilized on a probe carrier; a second step of spotting a liquid droplet containing a substrate for a luminescence reaction onto a location where a probe is immobilized on a probe carrier; and a third step of spotting a liquid droplet containing a sensitizer for a luminescence reaction onto a location where a probe is immobilized on a probe carrier.

Preferably, all of the above-mentioned first to third steps may be performed in the stated order. However, two of those steps may be performed or the steps may be performed in a different order.

Further, prior to spotting a liquid containing a reaction-contributing substance that contributes to a luminescence reaction, at least two or more selected from the group consisting of the luminescence reagent, the substrate for the luminescence reaction, and the sensitizer for the luminescence reaction may be mixed and the resulting mixture may be then spotted.

(3) Step of Detecting Luminescence Signal

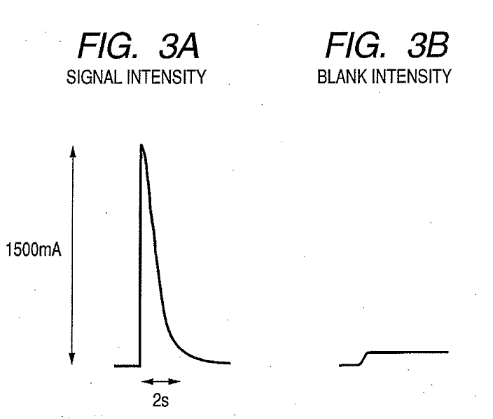

In the above-mentioned step (2), a luminescence reaction is initiated by spotting a luminescence reagent on the spot location. This reaction may proceed steeply or slowly depending on a reagent used. In general, however, the reaction proceeds steeply as represented by a diagram of a signal change with respect to elapsed time shown in FIG. 3A. That is, the reaction will complete within several microseconds. In addition, FIG. 3B shows a signal change in which the spotting of luminescence reagent does not initiate the luminescence reaction. In other words, the signal change in the absence of a target substance is shown. By detecting the difference in change, it becomes possible to attain detection (presence or absence and quantitative assay) with higher sensitivity, compared with the conventional fluorescence method.

It is preferable to sequentially spot the same luminescence reagent on a plurality of probe locations and observe luminescence signals depending on the timing of spotting. That is, the time of initiating the reaction is controlled by the timing of spotting, and the timing of detection may be also synchronized therewith. Therefore, in the case of a method of carrying out a head-scanning type spotting procedure as shown in FIG. 1, the detection means can also follow the spotting means and can employ a scanning type, thereby allowing simpler detection.

Note that an array sensor such as CCD or CMOS may be used to monitor the entire area, where all of probes are being fixed, over time. In this case, it is preferable to configure the device to output data in which the information about discharge timing and the results to be detected are coordinated.

### EXAMPLES

Hereinafter, the present invention will be described in more detail with reference to the following examples:

The detection of an enzyme was carried out using a device having the configuration shown in FIG. 1.

### Example 1

(1) Preparation of Substrate

A quartz glass substrate of 1 inch×3 inches (25.4 mm×76.2 mm) with a thickness of 1 mm was prepared. The quartz glass substrate was lightly washed with water, then immersed in a substrate-washing solution for ultrasonic cleaning for 20 minutes, and left standing all day and night. Subsequently, the substrate was pulled out and then washed with water and ultra pure water to wash out the washing solution, followed by being immersed in an aqueous solution of 1 M (mol/l) NaOH, which was previously heated to 60° C., for 20 minutes. The substrate was pulled out and then washed with water and ultra pure water to wash out the aqueous NaOH solution, followed by ultrasonic cleaning in ultra pure water for 20 minutes.

Subsequently, the substrate was immersed in a silane coupling agent (manufactured by Shin-Etsu Chemical, trade name: KBM603) for 1 hour. Here, the silane coupling agent was previously dissolved in water so as to have a concentration of 1% by mass and subjected to hydrolysis for about 1 hour. After lightly washing the substrate with ultra pure water, water droplets remained on the surface was flown out and dried by nitrogen gas and then heated in an oven to bake at 120° C. for 2 hours. Amino groups were hence introduced on the surface of glass because the silane coupling agent binds to the glass surface.

After that, EMCS (trade name, N-(6-maleimidocaproyloxy)succinimide, a cross-linking agent supplied by DOJINDO) was dissolved in a mixture solvent (ethanol:dimethyl sulfoxide)=1:1 (volume base)) at a ratio of 3 mg to 10 ml. The glass substrate previously baked was immersed in the obtained EMCS solution and left standing for 2 hours. After that, the substrate was pulled out of the EMCS solution and then lightly washed with the same mixture solvent as one used before, followed by replacing liquid droplets on the surface with ethanol and blowing out and drying the liquid droplets with nitrogen gas. Consequently, the substrate (EMCS substrate) having EMCS attached on the entire surface (both surfaces) of the substrate was obtained. The EMCS contains a succimide group and a maleimide group, and the succimide group is coupled with an amino group on the surface of the substrate, so the maleimide group can be introduced to the surface of the substrate.

(2) DNA Binding

A 25-mer modified DNA (probe) in which a thiol group (SH group) was bound to the terminal end was synthesized by BEX at our request. The substrate sequence of the modified DNA is the following sequence and the 5′-terminal thereof was bound with a SH group.

(Sequence) 5′HS-CGTACGATCGATGTAGCTAGCATGC-3′

A method of immobilizing probes was carried out on the basis of examples disclosed in Japanese Patent Application Laid-Open No. H11-187900.

(3) Hybridization

A 25-mer biotin-labeled DNA having a sequence complementary to the DNA bound to the substrate was synthesized by BEX at our request. The biotin-labeled DNA was dissolved in a 1 M NaCl/50 mM phosphate buffer solution (pH 7.0) to a final concentration of 1 μM, and 2 ml of the obtained solution was sealed in a hybridization package with the substrate, followed by carrying out a hybridization reaction for 3 hours. Subsequently, the substrate was washed with a 1 M NaCl/50 mM phosphate buffer solution (pH 7.0).

(4) Enzyme Labeling

Streptavidin-bound horseradish peroxidase was dissolved in a 1 M NaCl/50 mM phosphate buffer solution (pH 7.0) to a final concentration of 1 μM. Then, 2 ml of the obtained solution was dropped onto the substrate and then incubated for 1 hour at room temperature to carry out enzyme labeling using avidin-biotin binding. Subsequently, the substrate was washed with a 1 M NaCl/50 mM phosphate buffer solution (pH 7.0), thereby obtaining a substrate 1 as a measuring object.

(5) Chemiluminescence Reaction

Three different solutions; 5.0×10⁻² M luminol (manufactured by Wako Pure Chemical), a 1.0×10⁻² M hydrogen peroxide solution, and a 5.0×10⁻² M p-iodophenol solution, were poured into three solution tanks 4, respectively. Luminol was dissolved with an aqueous sodium hydroxide solution under alkaline conditions and then adjusted to pH 7.2 with a 3-(N-morpholino)propanesulfonic acid (MOPS) buffer solution. The p-iodophenol solution was also adjusted to pH 7.2 using the MOPS buffer solution. A prototype inkjet head was used to discharge a chemiluminescence reagent.

The inkjet head used includes a base board with which discharge orifices, liquid chambers for storing liquid, which are communicated with the discharge orifices, and heaters for applying discharge energies on the liquid in the liquid chambers are provided. When electric signals are applied on the heaters in the base board, an air bubble is be generated in the liquid adjacent to the heater, and then the pressure of the air bubble allows a liquid droplet to be discharged from the discharge orifice. The liquid droplet discharged flies to the surface of a solid phase. The amount of liquid, which can be discharged using the head having such a configuration, may be controlled to about 2 to 50 picoliters.

Alternatively, a commercially-available Bubble-Jet printer may be reconstructed. For example, a Bubble-Jet printer (trade name: BJF-850, manufactured by Canon) can be reconstructed to allow inkjet printing on a flat plate, and print patterns are then entered into the printer according to a predetermined file-making method. Therefore, it is possible to spot about 5 pl of a liquid droplet including reagent with a pitch of about 120 μm.

Further, an ink tank for Bubble-Jet printer was filled with the reagent and then mounted on the printing head. By using the reconstructed Bubble-Jet printer, an operation of spotting the reagent may be then carried out on the surface of a glass substrate.

(6) Detection of Chemiluminescence

As shown in FIG. 1, a detector 8 was fixed under a stage (mounting stage) 3 on which a substrate 1 is mounted, and the luminescence level was then detected immediately after dropping the chemiluminescence reagent on the substrate. A high-resolution digital B/W cold CCD camera manufactured by Hamamatsu Photonics (trade name: ORCA II-ER-1394) was used as the detector. The exposure time of the CCD camera was 10 seconds. As a result, the luminescence level obtained had a chemiluminescence strength of 100 and an S/N ratio of 20. In addition, 3σ (σ is a standard deviation) to the average value of the chemiluminescence strengths obtained by five measurements was 0.047. Consequently, the extremely high reproducibility of chemiluminescence strength was found.

### Example 2

An experiment was carried out in a similar manner as that of Example 1 except that 5.0×10⁻² M isoluminol (manufactured by Wako Pure Chemical) was used instead of 5.0×10⁻² M luminol. Consequently, the same results as those of Example 1 were obtained.

### Example 3

An experiment was carried out in a similar manner as that of Example 1 except that streptavidin-bound alkaline phosphatase was used instead of streptavidin-bound horseradish peroxidase. Consequently, it was confirmed that the streptavidin-bound alkaline phosphatase can also be suitably used in the present invention.

### Example 4

An experiment was carried out in a similar manner as that of Example 1 except that streptavidin-bound luciferase was used instead of streptavidin-bound horseradish peroxidase, and Intelite AB (trade name), that is a luminescence reagent kit manufactured by Kikkoman, was used as a luminescence reagent. Consequently, it was confirmed the enzyme and luminescence reagent can also be suitably used in the present invention.

### Example 5

(1) Preparation of Substrate

A quartz glass substrate of 1 inch×3 inches (25.4 mm×76.2 mm) with a thickness of 1 mm was prepared. The quartz glass substrate was lightly washed with water, then immersed in a substrate-washing solution for ultrasonic cleaning for 20 minutes, and left standing all day and night. Subsequently, the substrate was pulled out and then washed with water and ultra pure water to wash out the washing solution, followed by being immersed in an aqueous solution of 1 M NaOH, which was previously heated to 60° C., for 20 minutes. The substrate was pulled out and then washed with water and ultra pure water to wash out the aqueous NaOH solution, followed by ultrasonic cleaning in ultra pure water for 20 minutes.

(2) Preparation of Antigen-Antibody Complex

2 P82 l of a rabbit IgG sample solution (5.0 mg/ml) was dropped onto the substrate and the substrate was then completely dried at room temperature. The substrate was washed three times with phosphate buffered saline (PBS, pH 7.0), then added with a 3% by mass BSA (bovine serum albumin) solution as a blocking agent, an incubated at 37° C. for 2 hours. After washing the substrate three times with PBS, 5 μl of a peroxidase-bound goat anti-rabbit IgG antibody solution (5.0 mg/ml) was dropped onto a spot of the rabbit IgG, followed by incubating at 37° C. for 1 hour.

(3) Chemiluminescence Reaction and Detection of Chemiluminescence

A chemiluminescence reaction and a chemiluminescence detection were carried out in a similar manner as (5) and (6) of Example 1. Therefore, the obtained luminescence level had a chemiluminescence strength per spot of 140 and an S/N ratio of 25. In addition, 3σ to the average value of the chemiluminescence strengths obtained by five measurements was 0.076. Consequently, it was found that the reproducibility of chemiluminescence strength is extremely high.

The results thus obtained indicate that even when a chemiluminescence reagent is only dropped on a spot area where a DNA probe or protein is immobilized, a luminescence level sufficient for detection can be obtained. It indicates that the reaction area can be minimized to detect chemiluminescence on the flat substrate even though a reaction vessel such as a well is not formed on the substrate.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2005-348011, filed Dec. 1, 2005 which is hereby incorporated by reference herein in its entirety.

1. A method of detecting a target substance by a luminescence reaction using a probe carrier in which a plurality of probes capable of specifically binding to a target substance are immobilized on different locations on a surface of a substrate, comprising spotting a liquid micro-droplet respectively containing a reaction-contributing substance that contributes to a luminescence reaction on each of the locations where the probes are immobilized on the probe carrier; and detecting a luminescence signal on the plurality of probes where a liquid droplet is spotted based on the timing of discharging a liquid droplet.

2. A method according to claim 1, wherein:
the spotting includes nozzle for discharging a liquid containing the reaction-contributing substance; and the detection method further comprises spotting a liquid droplet individually on each of the locations where the probes are immobilized on the probe carrier by one nozzle according to a relative move of the nozzle to the probe carrier.

3. A method according to claim 1, wherein a probe carrier in which the plurality of probes is immobilized has a flat substrate.

4. A method according to claim 1, wherein the luminescence reaction comprises a luminescence reaction using an enzyme.

5. A method according to claim 1, wherein the spotting includes at least one of:
a first step of spotting a liquid droplet containing a luminescence reagent onto a location where a probe is immobilized on the probe carrier;
a second step of spotting a liquid droplet containing a substrate for a luminescence reaction onto a location where a probe is immobilized on the probe carrier; and
a third step of spotting a liquid droplet containing a sensitizer for a luminescence reaction onto a location where a probe is immobilized on the probe carrier.

6. A method according to claim 1, further comprising:
mixing at least two or more selected from the group consisting of a luminescence reagent, a substrate for the luminescence reaction, and a sensitizer for the luminescence reaction; and
spotting a liquid containing a mixture thereof.

7. A method according to claim 1, wherein the luminescence reaction is a chemiluminescence reaction or a bioluminescence reaction.

8. A method according to claim 1, wherein the probe is a nucleic acid, or one of an antigen and an antibody.

9. A method according to claim 1, wherein the luminescence signal is detected at the applied location on the substrate simultaneously with or immediately after the discharge timing.

10. A detecting device for detecting a target substance by a luminescence reaction using a probe carrier in which a plurality of probes capable of specifically binding to a target substance are immobilized on different locations on a surface of a substrate, comprising;
a spotting means spotting a liquid micro-droplet respectively containing a reaction-contributing substance that contributes to a luminescence reaction on each of the locations where the probes are immobilized on the probe carrier; and
a detecting means for detecting a luminescence signal on the probe location spotted based on the timing of discharging a liquid droplet.

11. A detecting device according to claim 10, wherein the spotting means is capable of controlling the application amount of the liquid and the application time thereof.

12. A detecting device according to claim 10, wherein the spotting means includes a head having discharge nozzles for discharging the liquid, and a tank for storing the liquid.

13. A detecting device according to claim 10, wherein the spotting means includes a plurality of tanks and a mixing vessel for mixing a plurality of reaction-contributing substances supplied from the plurality of tanks.

14. A detecting device according to claim 10, wherein the detecting means is capable of detecting a luminescence signal at an applied location on the substrate simultaneously with or immediately after the discharge of the liquid.

15. A detecting device according to claim 10, wherein the substrate of the probe carrier is transparent, and the detecting means has a structure capable of detecting the luminescence signal from the side opposite to the side of the substrate where the substance is applied.
